(19) United States
(12) Patent Application Publication
(10) Pub. No.: US 2023/0070144 A1
(43) Pub. Date: Mar. 9, 2023

(54) METHODS AND SYSTEMS FOR CONTROLLED MITOCHONDRIA TRANSFER

(71) Applicant: City University of Hong Kong, Hong Kong (CN)

(72) Inventors: Dong SUN, Hong Kong (CN); Adnan SHAKOOR, Hong Kong (CN)

(21) Appl. No.: 17/447,075

(22) Filed: Sep. 8, 2021

(51) Int. Cl. B01L 3/00

(52) U.S. Cl. CPC B01L 3/502761; B01L 2200/027; B01L 2200/0668; B01L 2300/06; B01L 2300/0861

(57) ABSTRACT

Systems and methods for automated optical tweezer (OT)-based mitochondrial transfer are provided. The system for automated optical tweezer-based mitochondrial transfer includes a microfluidic device and an optical tweezer micromanipulation system. The microfluidic device includes one or more confinement means for confining cells and a channel for flowing mitochondria near the confinement means. The optical tweezer micromanipulation system is configured to trap at least one of the mitochondria within the channel of the microfluidic device for transport of the mitochondria to one of the confined cells.

### TECHNICAL FIELD

The present invention generally relates to optical tweezer manipulation, and more particularly relates to methods and systems for quantity and quality control for mitochondria transfer in single cells using an optical tweezer micromanipulation system.

### BACKGROUND OF THE DISCLOSURE

Mitochondria are essential organelles known as the powerhouse of cells. Mitochondrial mutations play a key role in aging and neurodegenerative diseases, and they are known to be important regulators of cell death and survival. The malfunction of mitochondrial DNA (mtDNA) can lead to the net production of reactive oxygen species, which is related to various aging diseases, such as Alzheimer's disease or amyotrophic lateral sclerosis.

Mitochondrial transfer is considered a potential method to restore the metabolic functions of mitochondrial mutations. To replace endogenous mitochondria, isolated exogenous mitochondria could be injected into single cells by using microneedles. However, mitochondrial transfer by direct microinjection with a glass microneedle usually exhibits low efficiency of only 0.3%. Moreover, the microneedle-based cell-injection method has several limitations, such as clogging of the glass microneedle tip, physical damage to the cell, and limitation in repeated injection into the same cell. To address these problems, a thermal nanoblade method, which uses a >3 μm-diameter microneedle to prevent clogging, has been developed. This method uses pulsed laser-induced bubble cavitation to open holes in the cell membrane, followed by a synchronized fluid to pump mitochondria. However, the microneedle is placed only on the surface of the cell membrane and not tightly wrapped inside the cell membrane, so the injection efficiency of this method is only 2%-3%.

Endocytosis and microcytosis are natural cell-membrane-engulfing processes. Cells can absorb particles ranging in size from nanometers to several micrometers. Mitochondrial transfer through endocytosis is extensively used as a high-throughput mitochondrial transfer process. To improve the absorption efficiency of mitochondria, a method of forcing mitochondria into cells through centrifugation and pressure-driven methods have been proposed. To improve the transfer efficiency of mitochondria, a method of using mitochondria conjugated with magnetic beads followed by using an external magnetic field to press them into the cell has been proposed. With this method, however, magnetic beads are also transferred with mitochondria, which may damage the host cell.

Thus, there is a need for methods and systems to control the quality and quantity of isolated mitochondria before they are transferred to cells. In addition, there is a need for methods and systems which take into account mitochondrial heterogeneity, including mitochondrial functionality that represents another level of mitochondrial complexity and which avoid the transfer of debris and dead mitochondria which may harm the host cell. Furthermore, other desirable features and characteristics will become apparent from the subsequent detailed description and the appended claims, taken in conjunction with the accompanying drawings and this background of the disclosure.

### SUMMARY

According to at least one aspect of the present embodiments, a system for mitochondria transfer onto cells is provided. The system includes one or more confinement means for confining cells, means for locating mitochondria near the confinement means, and an optical tweezer micromanipulation system. The optical tweezer micromanipulation system is configured to trap at least one of the mitochondria for transport of the at least one of the mitochondria to one of the confined cells.

According to another aspect of the present embodiments, a method for transporting mitochondria to cells for absorption by the cells is provided. The method includes trapping the mitochondria by an optical tweezer micromanipulation system. The method further includes transporting the mitochondria over a confined cell and placing the mitochondria on a surface of the confined cell so that the confined cell can absorb the mitochondria by endocytosis.

According to a further aspect of the present embodiments, a computer readable medium containing program instructions for enabling transportation of mitochondria to cells by an optical tweezer micromanipulation system for absorption by the cells is provided. The program instructions when compiled into a processor are configured to cause the processor to trap a mitochondrion by the optical tweezer micromanipulation system, transport the mitochondrion/mitochondria over a confined cell, and place the mitochondrion/mitochondria on a surface of the confined cell so that the confined cell can absorb the mitochondrion/mitochondria by endocytosis.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying figures, where like reference numerals refer to identical or functionally similar elements throughout the separate views and which together with the detailed description below are incorporated in and form part of the specification, serve to illustrate various embodiments and to explain various principles and advantages in accordance with present embodiments.

FIG. 1, comprising FIGS. 1A to 1E, depicts illustrations of system setup and procedures of an automated optical tweezer (OT)-based mitochondrial transfer method in accordance with present embodiments, wherein FIG. 1A depicts an illustration of the system for controlling mitochondrial transfer to recipient cells using OT-based transport, FIG. 1B depicts an illustration of cell patterning and mitochondrial placement in the microfluidic device of FIG. 1A, FIG. 1C depicts an illustration of a mechanism and procedure of mitochondrial transport using the OT system, FIG. 1D depicts an illustration of an endocytosis process of mitochondrial absorption, and FIG. 1E depicts an illustration of dimensions of single-cell-confinement channels of the microfluidic device.

FIG. 2, comprising FIGS. 2A and 2B, depict the automated OT system in accordance with the present embodiments, wherein FIG. 2A depicts a photograph of an OT experimental setup and FIG. 2B depicts a block diagram of the automated OT system.

FIG. 3 depicts a flowchart of an automatic mitochondria-transport method in accordance with the present embodiments.

FIG. 4, comprising FIGS. 4A, 4B and 4C, depicts simulations of flow through the middle and cell-trapping channels of the microfluidic device for different fluid velocities in accordance with the present embodiments, wherein FIG. 4A depicts fluid flow of FIG. 4B depicts fluid flow of 2 μl/min, and FIG. 4C depicts fluid flow of 3 μl/min.

FIG. 5, comprising FIGS. 5A, 5B and 5C, depicts simulations of sheath flow and mitochondrial injection-flow rate in a microfluidic device in accordance with the present embodiments for a constant mitochondrial injection-flow rate in a first inlet of the microfluidic device and for different mitochondrial injection-flow rates in a second inlet, wherein FIG. 5A depicts mitochondrial injection-flow rate in the second inlet of 0.2 μl/min, FIG. 5B depicts mitochondrial injection-flow rate in the second inlet of 1 μl/min, and FIG. 5C depicts mitochondrial injection-flow rate in the second inlet of 1.5 μl/min.

FIG. 6, comprising FIGS. 6A, 6B and 6C, depicts experimental results of different fluid velocities in accordance with the present embodiments corresponding to the simulation results of FIGS. 4A, 4B and 4C, wherein FIG. 6A depicts experimental results of 293T cells in the microfluidic device of FIG. 1A after one hour of single-cell confinement, FIG. 6B depicts experimental results of adult mesenchymal stem cells (aMSCs) in the microfluidic device of FIG. 1A after three hours of single-cell confinement, and FIG. 6B depicts experimental results of mitochondria placement in the middle channel of the microfluidic device of FIG. 1A.

FIG. 7, comprising FIGS. 7A, 7B and 7C, depicts fluid velocity experimental results of yeast cells in the microfluidic device in accordance with the present embodiments, wherein the experimental results of FIGS. 7A, 7B and 7C correspond to the simulation results of FIGS. 5A, 5B and 5C.

FIG. 8, comprising FIGS. 8A and 8B, FIG. 9, comprising FIGS. 9A and 9B, and FIG. 10, comprising FIGS. 10A and 10B, depict different numbers of mitochondrial trapping with the systems and methods in accordance with the present embodiments.

FIG. 11 depicts image processing for the detection of functional mitochondria of fetal mesenchymal stem cells (fMSCs) in accordance with the present embodiments, wherein image 1 depicts isolated functional and non-functional mitochondria, image 2 depicts a threshold image with white regions indicating the functional mitochondria, image 3 depicts a smoothed image having noise removed, image 4 depicts edge detection and mitochondrial size calculation, image 5 depicts mitochondria selected based on functionality and size, and image 6 depicts a result of the mitochondria selection process merged with the original image (image 1).

FIG. 12, comprising FIGS. 12A to 12D, depicts single-cell detection in accordance with the present embodiments, wherein FIG. 12A depicts a template image, FIG. 12B depicts an aMSC confined in the U-shaped microfluidic channel, FIG. 12C depicts an image obtained by edge detection, and FIG. 12D depicts a final detected area of the image through the template-matching algorithm.

FIG. 13, comprising FIGS. 13A and 13B, depicts confinement of cells in the one-dimensional array of U-shaped channels within the microfluidic device in accordance with the present embodiments, wherein FIG. 13A depicts an image taken under a bright field fluorescence microscope and FIG. 13B depicts an image taken under a dark field fluorescence microscope.

FIG. 14, comprising FIGS. 14A to 14F, depicts an experimental sequence of trapping, transporting and transferring a single mitochondrion into a cell in accordance with the present embodiments, wherein FIG. 14A depicts the confined cell, FIG. 14B depicts detecting and trapping a single mitochondrion, FIG. 14C depicts locating a center of the U-shaped cell corresponding to a center of the confined cell, FIG. 14D depicts aligning the mitochondrion above the cell, FIG. 14E depicts placing the mitochondrion on the surface of the cell, and FIG. 14F depicts the mitochondrion absorbed by the cell through endocytosis approximately one hour after being placed on the surface of the cell.

FIG. 15, comprising FIGS. 15A, 15B and 15C, depicts three-dimensional reconstruction of cells after transfer of mitochondria into single 293T cell in accordance with the present embodiments, wherein FIG. 15A reconstruction of the cell after transfer of one mitochondrion, FIG. 15B reconstruction of the cell after transfer of four mitochondria, and FIG. 15C reconstruction of the cell after transfer of six mitochondria.

FIG. 16, comprising FIGS. 16A to 16F, depicts an experimental sequence of trapping, transporting, and transferring three fMSC-isolated mitochondria to an aMSC in accordance with the present embodiments, wherein FIG. 16A depicts the confined cell, FIG. 16B depicts detecting and trapping three mitochondria, FIG. 16C depicts bringing the three mitochondria close together to facilitate transportation and raising them, FIG. 16D depicts aligning the three mitochondria above the cell, FIG. 16E depicts placing the three mitochondria on the surface of the cell, and FIG. 16F depicts successful transfer of the two mitochondria to the cell pointed with white arrows.

FIG. 17, comprising FIGS. 17A and 17B, depicts confocal images showing six fMSC-isolated mitochondria transferred inside an aMSC in accordance with the present embodiments, wherein FIG. 17A depicts the endogenous mitochondria in the aMSC stained with MitoTracker Green and FIG. 17B depicts the red transferred mitochondria pointed with white arrows within the endogenous mitochondria evidencing successful endocytosis.

FIG. 18, comprising FIGS. 18A and 18B, depict bar graphs of experimental results for two types of cells (aMSCs and 293T cells) in accordance with the present embodiments, wherein FIG. 18A depicts a bar graph of mitochondrial transfer efficiency of the two types of cells with different confinement-channel heights of the microfluidic device and FIG. 18B depicts a bar graph of cell viability of the two types of cells after mitochondria transfer.

FIG. 19, comprising FIGS. 19A to 19D, depicts graphs of experimental results of varying parameters of the automated optical tweezer-based mitochondria-transfer system in accordance with the present embodiments, wherein FIG. 19A depicts the relationship between single-cell hydrodynamic confinement efficiency and inlet flow rate, FIG. 19B depicts the relationship between mitochondrial viability and different optical tweezer powers and trapping times, FIG. 19C depicts the relationship between mitochondria-transport efficiency and laser power, and FIG. 19D depicts the relationship between mitochondrial transport efficiency and transporting speed. Error bars represent standard deviations in all cases.

FIG. 20 depicts images showing the effect of optical trapping in accordance with the present embodiments on the functionality of fMSC-isolated mitochondria at different laser powers and exposure times.

FIG. 21 depicts confocal and fluorescence microscopy images showing quality control of mitochondrial transfer using a conventional co-culture method and using the optical tweezer-based methods in accordance with the present embodiments.

FIG. 22, comprising FIGS. 22A and 22B, depicts quantity control of mitochondrial transfer achieved using the optical tweezer-based mitochondrial-transfer system and methods in accordance with the present embodiments, wherein FIG. 22A depicts confocal three-dimensional images of different numbers of fMSC-isolated mitochondria transferred in 293T cells and FIG. 22B depicts confocal three-dimensional images of different numbers of 293T-isolated mitochondria transferred in aMSCs.

FIG. 23 depicts images showing quantity control of mitochondrial transfer using a conventional co-culture method and using the optical tweezer-based methods in accordance with the present embodiments.

And FIG. 24, comprising FIGS. 24A to 24D, single-cell qPCR results of single aMSC transferred with different numbers of fMSC's isolated mitochondria, wherein FIG. 24A depicts expression level of TERT, FIG. 24B depicts expression level of P16, FIG. 24C depicts expression level of PGK1, and FIG. 24D depicts expression level of DLST.

Skilled artisans will appreciate that elements in the figures are illustrated for simplicity and clarity and have not necessarily been depicted to scale, and that the number in the graphs may have been normalized for simplicity and clarity.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description. It is the intent of present embodiments to present unique methods and systems for controlling the quality and quantity of mitochondria transferred into single cells by using an automatic optical tweezers micromanipulation system. The systems in accordance with the present embodiments can automatically, accurately, and efficiently collect and transport healthy mitochondria to cells, and the recipient cells then take up the mitochondria through endocytosis. In addition, in accordance with the present embodiments microfluidic devices are developed to pattern the cells and mitochondria, and then the automatic optical tweezers micromanipulation system is used to transport a predefined number of mitochondria to the cells.

In aging-related diseases, onsite senescent cells contribute to the degeneration of function through the senescence-associated secretory phenotype (SASP). It has been demonstrated that human fetal mesenchymal stem cells' (fMSCs′) secretome could ameliorate the senescent phenotype of human adult mesenchymal stem cells (aMSCs) in in-vitro culture and in-vivo model. These findings suggest that fMSCs may have a unique metabolite profile in the secretome and the ability of anti-senescence. Given that mitochondria are well-known metabolic regulators, one of the advantages of the present embodiments is the ability to transform aMSCs into fMSC-like cells through mitochondrial transfer.

Optical tweezers are a unique tool that utilizes a highly focused light beam to provide a trapping force in the order of piconewtons. Optical tweezers have been used for many biological tasks such as mitochondria trapping and cell transport because of their advantage for manipulating trapped particles noninvasively, flexibly, and precisely. In accordance with the present embodiments, several robotic strategies have been developed to automate cell manipulation with optical tweezers. Also, several microfluidic devices have been developed as cell-processing platforms to simplify biological operations, such as single-cell manipulation and analysis.

A new micromanipulation system and methods have been developed in accordance with the present embodiments that integrate optical tweezers, microfluidic devices, and automation technologies to achieve precise and automated transport of isolated mitochondria to single cells for the transfer of mitochondria from fMSC (young) to aMSC (old) to improve the senescence of aMSC (old). The microfluidic device in accordance with the present embodiments patterns the cells and mitochondria, and then the optical tweezers in accordance with the present embodiments is used to transport a predefined number of the mitochondria to the cells, the cells absorbing the mitochondria through endocytosis.

Existing mitochondria transfer methods ignore mitochondrial heterogeneity, including mitochondrial functionality. The methods and systems in accordance with the present embodiments enable automatic and precise collection and transport of healthy mitochondria onto cells with high efficiency, where the cells uptake these healthy mitochondria through endocytosis. The number of mitochondria to be transferred is an important aspect in precision medicine. The exact number of mitochondria that could cure or cause a significant change in single cells for a particular disease is completely unknown. Thus, precise quality-controlled mitochondrial transfer methods such as the methods in accordance with the present embodiments are exceedingly essential. The mitochondrial transfer methods in accordance with the present embodiments allow predefined number of mitochondria to be accurately collected, transported and transferred to each individual cell, depending on the available optical tweezer operating system.

The automatically controlled optical tweezers are controlled in accordance with the present embodiments to automatically and precisely collect and transport an accurate number of the mitochondria to the cells with high efficiency. Mitochondria isolated from fMSCs can be transferred to aMSCs and 293T kidney cells, indicating that the transfer methods in accordance with the present embodiments can be applied to different cell types.

In accordance with the present embodiments, the effect of mitochondrial transfer from fMSCs to aMSCs was studied by performing quantitative polymerase chain reaction (qPCR) analysis on single cells after the mitochondrial transfer. The results show that the transfer of isolated mitochondria from fMSCs to aMSCs can significantly increase the anti-aging and metabolic gene expression levels of aMSCs, thereby transforming aMSC into fMSC-like phenotype.

Existing mitochondria transfer methods ignore mitochondrial heterogeneity, including mitochondrial functionality that represents another level of mitochondrial complexity. Compared with existing mitochondria transfer techniques, mitochondrial transfer methods in accordance with the present embodiments advantageously detects only healthy isolated mitochondria using image processing and then transfers them to specific single cells.

The number of mitochondria to be transferred is an important aspect of precision medicine. Compared to existing mitochondria transfer methods, the mitochondria transfer method in accordance with the present embodiments advantageously enable a predefined number of mitochondria to be accurately collected, transported and transferred to each cell. The development of a precise quantity-controlled mitochondria transfer method such as the systems and methods in accordance with the present embodiments answers questions such as: How many transferred mitochondria will be enough to make a significant change in a single cell?

The automated optical tweezer manipulation method in accordance with the present embodiments precisely controls the quantity and quality of mitochondria before transfer and the mitochondria from fMSCs can be used as a potential source to reverse the aging-related phenotype and improve metabolic activity in aMSCs. Accordingly, the methods and systems in accordance with the present embodiments will substantially benefit precision medicine and cell therapy in mitochondrion-related diseases.

As the field of precision medicine develops, it requires ever-increasing technological advancements across a wider range of disciplines. The methods and systems in accordance with the present embodiments can be extended to transfer other small organelles or cargoes such as lysosomes, bacteria, and microbeads into cells. The precise transfer of different microscale organelles allows researchers to further study the function of each organelle after transfer in a single cell. Additionally, optical tweezer systems can manipulate various nano- and micro-sized objects, so the systems and methods in accordance with the present embodiments can be used to study the behavior of cells receiving these tiny objects as drug delivery, as well as to study their significance in precision medicine. In addition, the methods and systems in accordance with the present embodiments validate the feasibility of mass producing function-altered cells by integrating robotics and manufacturing technologies into cell manipulation. Successful production of therapeutic quantities of feature-improved cells will lead to broad and varied applications in biopharmaceuticals, gene and cell therapies, and tissue engineering.

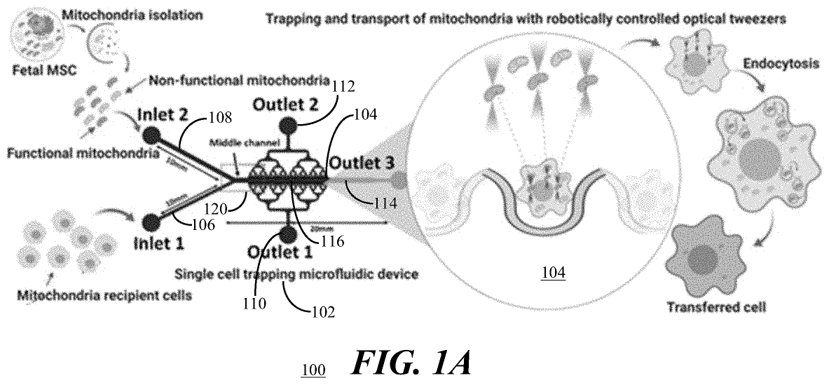

Referring to FIG. 1A, an illustration 100 depicts an automated optical tweezer manipulation approach in accordance with the present embodiments which is developed for mitochondrial transfer in single cells. A microfluidic cell-positioning device 102 is used to pattern cells and mitochondria and an OT manipulation system 104 is used to automatically collect a predefined number of healthy isolated mitochondria and transport them onto the top of the cells in accordance with the present embodiments. The cells then absorb the transferred mitochondria through endocytosis. In contrast to other mitochondria transfer methods, the system and method in accordance with the present embodiments efficiently controls the quantity and quality of the mitochondria before transfer, thereby providing advantageous and beneficial solutions including solutions which can considerably contribute to precision medicine and cell therapy of mtDNA-related diseases.

The microfluidic device 102 is designed to process the transport of mitochondria to cells and includes two inlets 106, 108, three outlets 110, 112, 114, and a central flow channel or middle channel 116. A portion 120 of the middle channel 116 is depicted in FIG. 1B. The microfluidic device 102 is fabricated in accordance with the present embodiments using soft lithography technology. The inlets 106 and 108 (Inlet 1 and Inlet 2 in FIG. 1A) are used to introduce recipient cells and isolated mitochondria, respectively, into the microfluidic device 102. The outlets 110, 112 (Outlet 1 and Outlet 2 in FIG. 1A) are designed as two tree-like structures with one hundred twenty-eight single-cell-confinement channels connected to the central flow channel 116 as terminals. The single-cell-confinement channels are geometrically-shaped confinement shape, wherein the shape and dimensions of the geometric shape are designed to correspond to the dimensions and shapes of cells to be confined. In accordance with the present embodiments, the single-cell confinement channels are U-shaped. The U-shaped single-cell-confinement channels are designed to be open on one side to allow cells to enter and closed on the other side to prevent cells and isolated mitochondria from escaping. The U-shaped single-cell-confinement channels (shown in more detail in FIGS. 1C and 1E) were patterned in a one-dimensional array which, in accordance with the present embodiments, significantly reduces the complexity of cell detection and automated mitochondrial transport. Outlet 1 110 is used for cell confinement and Outlet 2 112 is used to generate uniform fluid flow in the middle channel 116 of the microfluidic device 102. The third outlet 114 (Outlet 3 in FIG. 1A) is used to collect cells from the microfluidic device for further analysis of mitochondrial-transfer cells. The middle channel is designed to place isolated mitochondria at a distance of about 100 μm from the cell to prevent mitochondria from interacting with the cell.

FIG. 1B depicts a magnified view of the portion 120 of the middle channel 116 of FIG. 1A in accordance with the present embodiments. Trapped mitochondrial receptor cells 122 are patterned in a one-dimensional array of hydrodynamic single-cell-confinement channels 124 and isolated mitochondria 126 are placed near the cells. A portion 128 of the single-cell confinement channels 124 are shown in a magnified view in FIG. 1C and a portion 130 of the single-cell confinement channels 124 are shown in a greatly magnified view in FIG. 1E.

Referring to FIG. 1C, the magnified view of the portion 128 of the single-cell confinement channels 124 depicts healthy mitochondria 132 identified by the imaging process and captured 134 by the optical tweezer system 104. The system 104 then, in accordance with the present embodiments, moves an X-Y stage to transport 136 the captured mitochondria 134 across the region 137 onto a surface 138 of a selected cell 140. Referring to FIG. 1D, a magnified illustration of the process within the region 137 depicts the healthy mitochondria 132 captured 134 by the optical tweezer system 104 being transported 136 onto the surface 138 of the selected cell 140 trapped in the single-cell confinement channel 124 and absorbed by the cell 140 through endocytosis, as shown in the process 150a, 150b, 150c. This process is repeated until all cells are processed. The dimensions of the single-cell-confinement channels 124 are shown in FIG. 1E in the greatly magnified view of the portion 130 of FIG. 1B.

Through Comsol Physics simulations and comprehensive experiments as discussed hereinbelow, the design, size, and shape of the microfluidic device 102 as well as operational parameters of the mitochondria transfer process have been optimized for the automated optical tweezer-based mitochondrial transfer system in accordance with the present embodiments. The automated optical tweezer-based mitochondrial transfer designed system has been tested for mitochondria transfer among different receptor cells in accordance with the present embodiments. For example, mitochondria isolated from fMSCs and 293T kidney cells were transferred to aMSCs and 293T kidney cells indicating that the mitochondria transfer method in accordance with the present embodiments can be applied to different cell types. Experimental results effectively demonstrate that, unlike previous mitochondrial transfer methods that ignore the functional status of isolated mitochondria, the mitochondria transfer method in accordance with the present embodiments performs quality and quantity control of mitochondria before transfer.

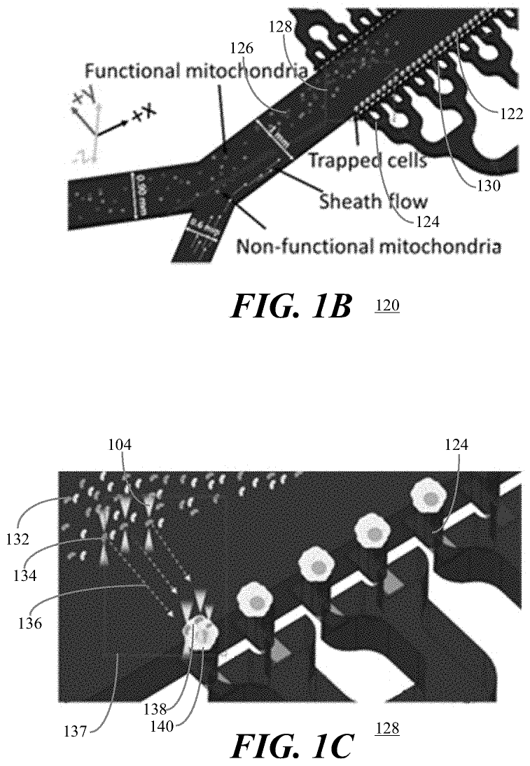

Referring to FIGS. 2A and 2B, a photograph 200 (FIG. 2A) and a block diagram 250 (FIG. 2B) schematically illustrate the automated optical tweezer system in accordance with the present embodiments. An exemplary system in accordance with the present embodiments contains three modules including an execution module 205, a control module 210 and a sensing module 215. The execution module 205 includes the optical tweezer system 104 (a holographic optical tweezer device used to create multiple traps (such as the Bioryx200 made by Arryx)), the microfluidic device 102 (as described hereinabove in regards to FIGS. 1A to 1E) to pattern cells and mitochondria, and two motorized positioning stages 224, 226 (such as Prior Scientific's ProScan) for movement in the X-Y plane and along the Z-axis. The X-Y stage 224 can move the microfluidic device 102 horizontally, and the Z-axis stage 226 can move an objective lens 228 below the X-Y stage 224 vertically. The execution module also includes two syringe pumps 230, 232 (such as the LSP01-2A, long pump) for injecting cells and mitochondria into the two inlets 106, 108 (FIG. 1A) of the microfluidic device 102 under control of a pump controller 234 (FIG. 2A).

The control module 210 includes a motion controller 240 for controlling the three degrees of freedom (DOF) of the motorized positioning stages 224, 226, the pump controller 234 (FIG. 2A) and a controller for the optical tweezer system 220, the controllers coupled to a computer 242 for automated control of the system in accordance with the present embodiments.

The sensing module 215 contains a microscope (e.g., Nikon TE2000) with the objective lens 228 (e.g., a 60× objective lens such as the CFI PLAN APO VC 60X/1.20 water immersion lens made by NIKON) and a CCD camera 244 (such as the FO124SC made by Foculus). The system was additionally equipped with an environmental-control system (TC-L-Z003, Live Cell Instrument, Inc.) to maintain a working condition of 37° C. and 5% CO₂ and an illumination lamp 246.

The optical tweezer system 104 uses a continuous-wave laser beam with a wavelength of 1064 nm and a maximum output power of 3 W. The laser beam reaches the objective lens 228 after being reflected by a dichroic mirror (DM1), thereby forming a three-dimensional optical trap. The forces and deformations exerted by the optical tweezer system 104 on trapped particles is in the orders of piconewtons (e.g. pN, 10⁻¹²N) and nanometers (nm, 10⁻⁹ m), respectively. A graphical user interface (GUI) interface for user interaction with the computer 242 was established using Visual C++, and an anti-vibration table 248 supported the mechanical setups.

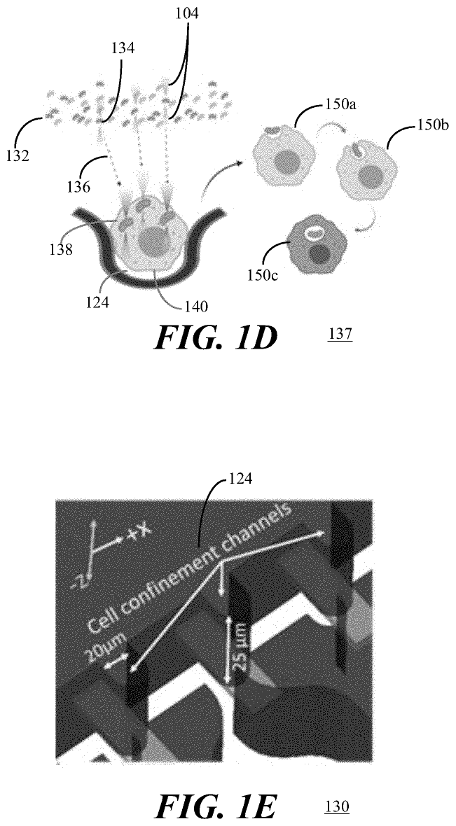

Referring to FIG. 3, a flowchart 300 depicts an automatic mitochondria-transport method in accordance with the present embodiments. The method begins by defining 302 the number of mitochondria to be trapped. The first cell is then brought into the field of view 304 to define the Z stage 226 and define the home location of the X-Y stage 224.

The stage is moved 100 μm along the +Y axis 306 to bring the mitochondria into the field of view. If no mitochondria is detected 308, step 306 is repeated until mitochondria is detected. When the mitochondria is detected 308, the mitochondria is trapped 310 and moved 20 μm along the +Z axis 312 to raise the mitochondria upward at a height more than the height level of the cell. The stage is then moved 100 μm along the −Y axis 314 until a cell is detected 316. When the cell is detected 316, the stage is moved to align the single-cell confinement channel 124 and the mitochondria 318. The trapped mitochondria is moved downward and released 320. The stage is then moved 60 μm along the +X axis 322 to bring the next cell into view.

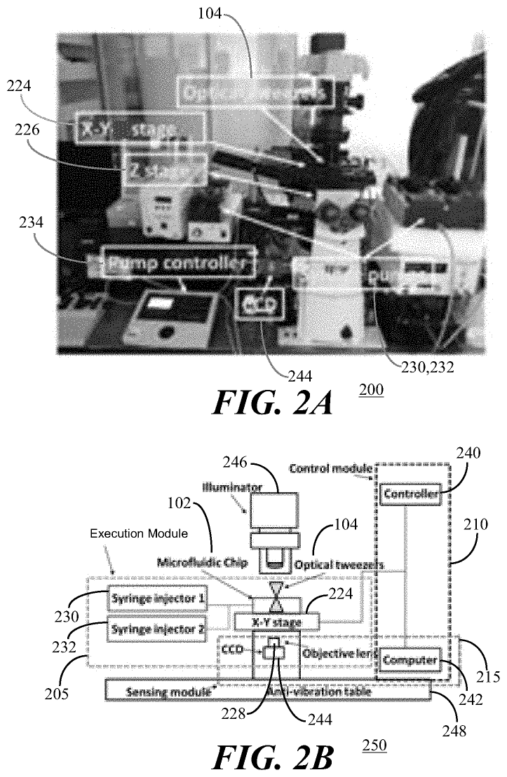

Comsol Physics simulations were utilized to optimize the design, size, and shape of the microfluidic device 102, as well as measure fluid velocities. To optimize single cell confinement, the simulation varied the fluid velocity in Inlet 1 106 of the microfluidic device 102 from 0.2 μL/min to 4 μL/min, and flow lines were observed through the middle and U-shaped cell-confinement channels. Referring to FIGS. 4A, 4B and 4C, illustrations 400, 430, 460 depict fluid velocities of 1 μl/min, 2 μl/min and 3 μl/min, respectively, in the middle and U-shaped cell-confinement channels of the microfluidic device 102.

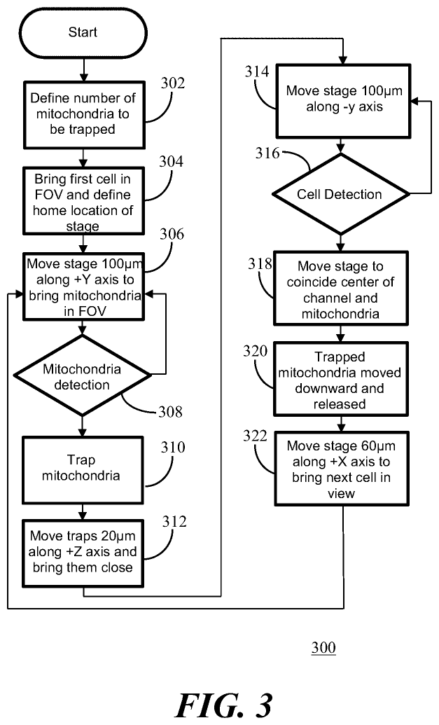

Similarly, to optimize mitochondria placement in the microfluidic device 102, different flow rates through Inlet 1 106 and Inlet 2 108 were applied to determine sheath flow and mitochondrial injection-flow rate in the microfluidic device 102. Referring to FIGS. 5A, 5B and 5C, illustrations 500, 530, 560 depict simulations of sheath flow in the inlets 106, 108 and the central flow channel 116 in the microfluidic device 102 when the mitochondrial injection-flow rate in the Inlet 1 106 is 0.5 μl/min and the mitochondrial injection-flow rate in the Inlet 2 108 is 0.2 μl/min (illustration 500), 1 μl/min (illustration 530) and 1.5 μl/min (illustration 560).

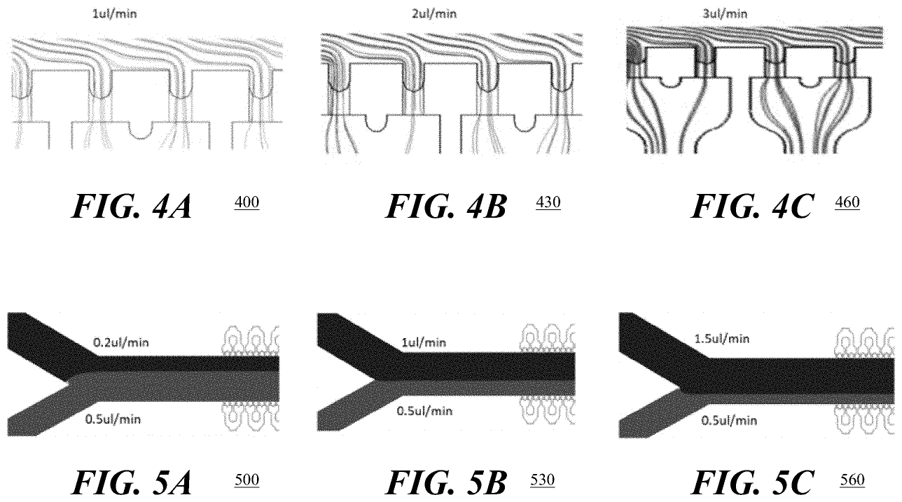

Single cell confinement in the microfluidic device 102 was experimentally confirmed. The microfluidic device 102 was attached to a glass slide by plasma bonding and three polyether tubes were inserted into the three outlets 110, 112, 114 of the microfluidic device 102 from one end and into a 1.5 mL tube from the other end. The microfluidic device 102 was then connected to a glass-slide holder fixed on the X-Y positioning stage 224. Recipient cells and isolated mitochondria were filled in two separate 1 mL syringes. The backsides of the syringes were connected to injectors and the front sides were connected using polyethene tubing to Inlet 1 106 and Inlet 2 108 of the microfluidic device. To obtain a one-dimensional array of single-cell confinement, the Inlet 2 108 and the Outlet 2 112 were initially blocked with metal clips to prevent cells from flowing into them. The syringe connected to the Inlet 1 106 was then opened at a flow rate of 1 μL/min to introduce cells into the microfluidic device 102. Within a few minutes, single cells were confined to the 128U-shaped cell-confinement channels 124 of the Outlet 1 110. Referring to FIGS. 6A and 6B, the experimental results 600, 630 depict 293T cells and aMSCs cells in the microfluidic device 102 after one hour and three hours of single-cell confinement, respectively.

Mitochondria placement in middle channel 116 of the microfluidic device 102 was achieved using fMSC-isolated mitochondria placed in the middle channel 116 and aMSC confined in the cell-confinement channels 124. The Inlet 2 108 and the Outlet 2 112 were unobstructed. The Inlet 1 106 was then changed to sheath flow by buffering with a buffer media (such as αMEM without calcium provided by Thermofisher) to keep mitochondria away from the one-dimensional array of confined cells during mitochondrial flow in the microfluidic device 102. The buffer media and mitochondria were introduced into the microfluidic device 102 through the Inlets 1 and 2 at flow rates of 0.5 μL/min and 1 μL/min, respectively, for two minutes. At these flow rates, mitochondria maintained a distance of approximately 100 μm from the one-dimensional confined cell array, as shown in the experimental results 660 (FIG. 6C).

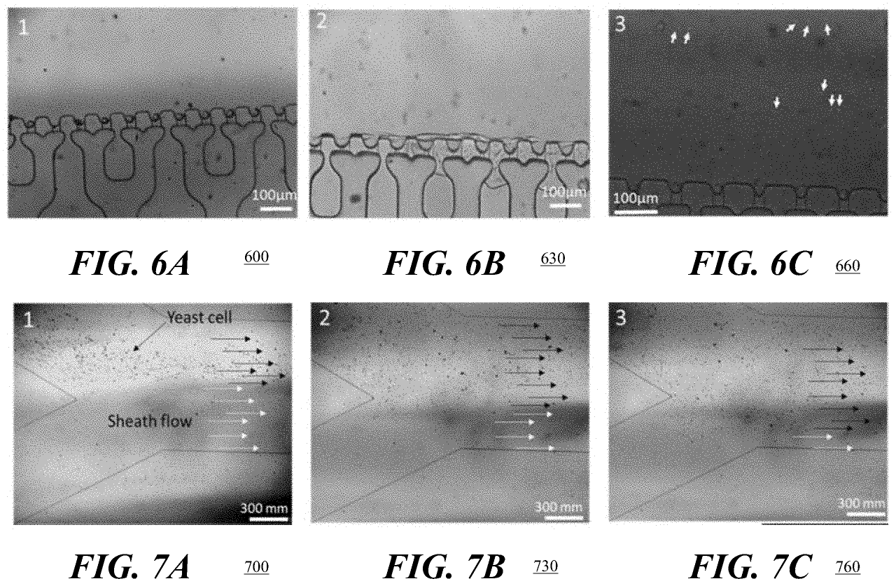

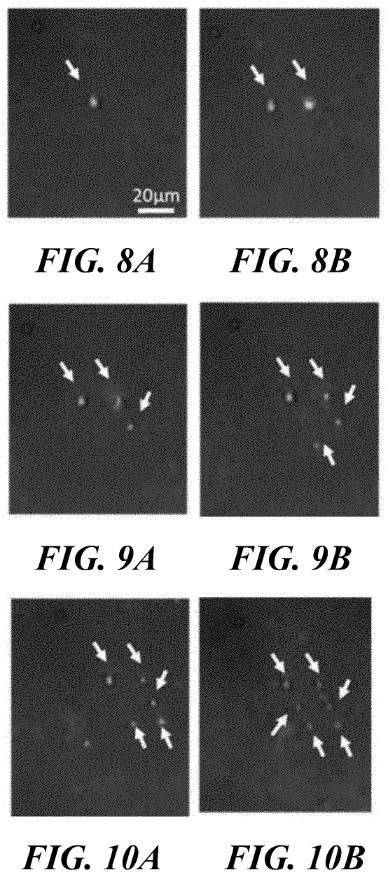

To test different fluid velocities experimentally, yeast cells were used because for single-cell confinement experiments, it is difficult to observe mitochondria on a low-magnification lens (4×). Referring to FIGS. 7A, 7B and 7C, experimental results 700, 730, 760 depict the experimental results using yeast cells corresponding to the simulation results 500, 530, 560. FIGS. 8A, 8B, 9A, 9B, 10A, and 10B depict different numbers of mitochondrial trapping with the systems and methods in accordance with the present embodiments as indicated by the arrows.

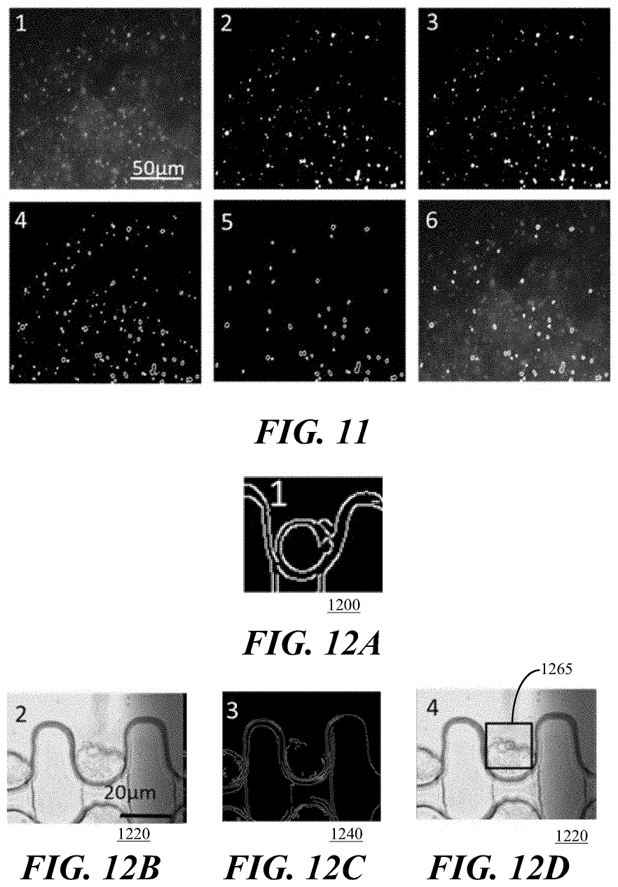

Healthy isolated mitochondria were detected using image processing. Image 1 of FIG. 11 shows a captured fluorescent image of isolated mitochondria stained with the membrane-potential dye JC-1 before isolation. The image processing algorithm in accordance with the present embodiments automatically distinguishes orange/red, yellow, and green mitochondria as shown in the image 1. By setting the range of a hue saturation value (HSV) to 10-25 for H, 100-255 for S, and 20-255 for V, a threshold image can be obtained from the original image where a white region represents a corresponding position of orange/red mitochondria as shown in image 2 of FIG. 11. Red or orange mitochondria detected within these HSV ranges can be regarded as high-membrane-potential mitochondria. For specific brightness settings (e.g., 100), exposure time settings (e.g., 400 ms), and gain value settings (e.g., 200), these HSV values remained unchanged. As shown in image 3 of FIG. 11, a median filter can be applied to minimize pepper and high-frequency noise in the image. This enables the contours of the white regions to be detected as shown in image 4, and the central moments of these contours can be calculated to obtain the positions of the white regions—i.e., the corresponding mitochondria. The mitochondria of relatively larger sizes were sorted, as shown in images 5 and 6 of FIG. 11. The distance of each sorted mitochondria to the center of the cell-confinement channels 124 containing the target recipient cells can then be calculated. A user-defined number of mitochondria can then be selected among the detected mitochondria and, after detection, the optical tweezer system 104 can be used to transport the detected mitochondria to the targeted cells.

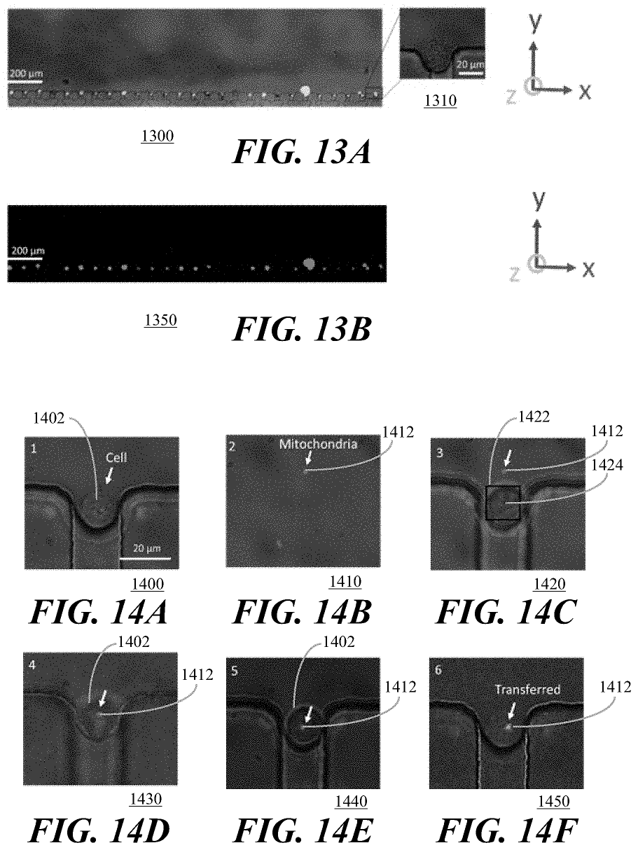

Cells have irregular shapes according to their suspension or adhesion state, so image-processing technology can be used to detect cells. The advantage of the one-dimensional array of the U-shaped cell-confinement channels 124 in accordance with the present embodiments is that the center of the channel can simply be used as the center of the detection cell, greatly simplifying the cell-detection process. Referring to FIG. 12A, an image 1200 shows the use of a template-matching algorithm used to detect the center of the U-shaped channels 124 in accordance with the present embodiments. Referring to FIG. 12B, a CCD captured an image 1220 of the channel containing the target cell and, as shown in FIG. 12C, converted a grayscale image of the image 1220 into a binary image 1240. The template image is then used to match the area of the U-shaped channel 124. As shown in FIG. 12D, after matching, a rectangle 1265 is drawn on the detected area within the image 1220, and the center of the rectangle 1265 is selected as the final location of the cell.

### EXAMPLES

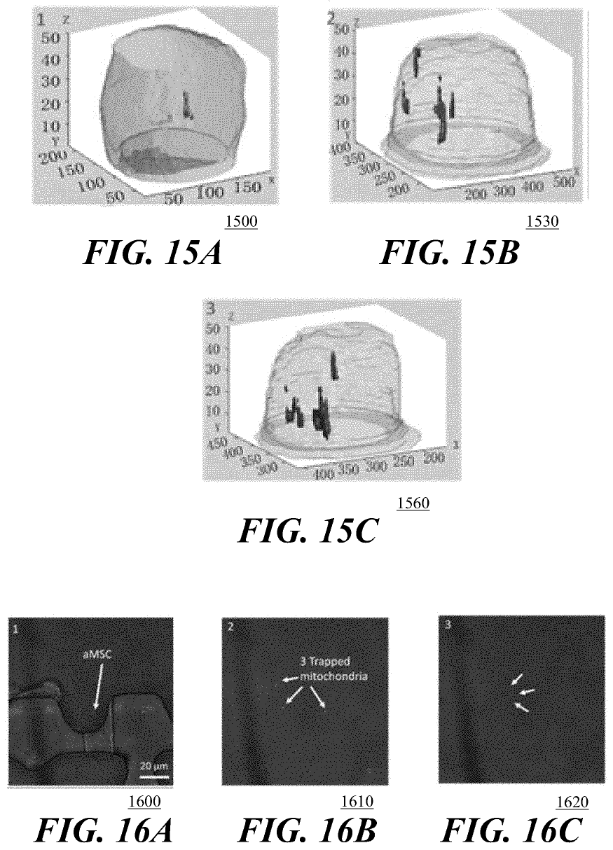

The mitochondrial transfer results with the automated optical tweezer manipulation system and methods in accordance with the present embodiments are illustrated hereinafter. For the experimental results, mitochondria were isolated from fMSCs and 293T kidney cells and then transferred to two different cell types, namely, 293T cells and aMSCs. Referring to FIGS. 13A and 13B, illustrations 1300, 1350 depict confinement of individual GFP-stained 293T cells in the one-dimensional array of U-shaped channels 124 within the microfluidic device 102 in accordance with the present embodiments. The illustration 1300 shows an image of the array of U-shaped channels 124 taken under a bright field fluorescence microscope with a 10× magnified image 1310 showing a single GFP-stained 293T cell. The illustration 1350 shows an image of the array of U-shaped channels 124 taken under a dark field fluorescence microscope.

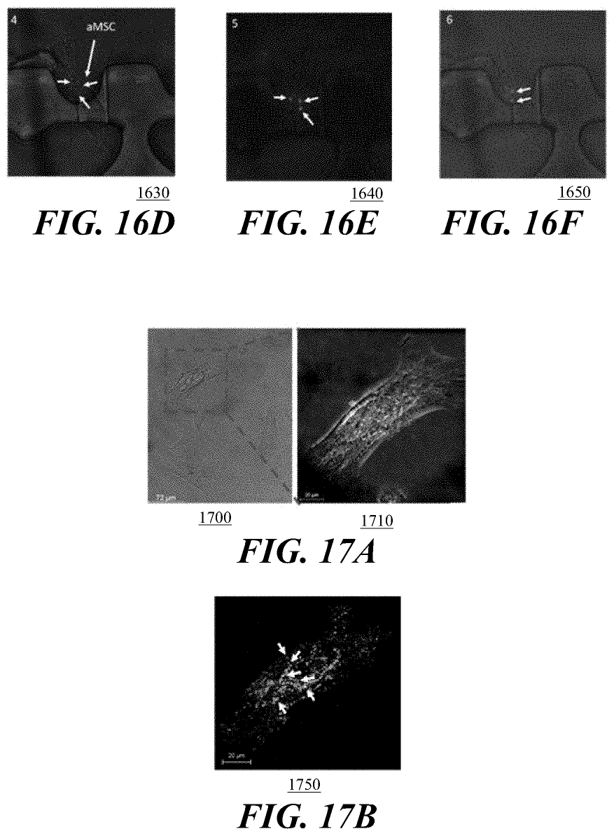

Referring to FIGS. 14A to 14F shows an experimental sequence corresponding to methods in accordance with the present embodiments. First, as shown in an image 1400 of FIG. 14A, a home position of the X-Y stage 224 is defined by bringing a first 293T cell 1402 confined in the microfluidic channel 116 into the field of view (FOV). After saving the Y coordinate of the current stage position as the home position, the stage 224 is moved 100 μm along the +Y axis to bring the isolated mitochondria into the FOV. Referring to FIG. 14B, an image 1410 depicts detection of a single mitochondrion 1412 with an image-processing algorithm and trapping of the mitochondrion 1412 by the optical tweezer system 104. The objective lens 228 is then automatically moved up 20 μm along the Z axis, making the height of the trapped mitochondrion 1412 greater than the height of the cell 1402 (e.g., a diameter of approximately 16-19 μm). Afterwards, the stage 224 is moved along the Y axis to reach its original position in the FOV, as shown in an image 1420 of FIG. 14C. As can be seen in the image 1420, the system uses image processing to detect the center of the U-shaped channel (i.e., the center 1424 of the rectangle 1422) to identify the cell location as discussed hereinabove. Then, the stage 224 is moved along the X and Y axes to align the trapped mitochondrion 1412 with the center 1424 of the U-shaped channel, as shown in an image 1430 of FIG. 14D. After alignment, the optical tweezer trap is moved 20 μm down along the Z axis, placing the mitochondrion 1412 on the top of the cell 1402 as shown in an image 1440 of FIG. 14E. The optical tweezer system 104 is then turned off. During the downward movement of the optical trap, the mitochondrion 1412 remains on top of the cell membrane (i.e., the surface of the cell 1402). After appropriately one hour, the mitochondrion 1412 is absorbed by the cell 14023 through endocytosis, as shown in an image 1450 of FIG. 14F.

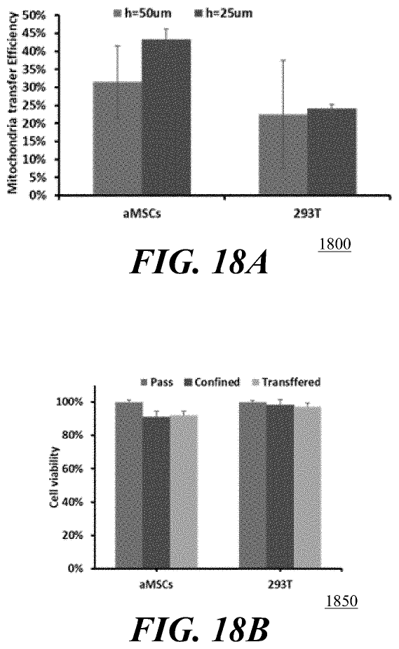

In FIGS. 15A, 15B and 15C, the three-dimensional graphs 1500, 1530, 1560 illustrate the three-dimensional reconstruction of the transferred 293T cell. The graph 1500 depicts reconstruction of the cell after transfer of one mitochondrion. Using the holographic optical tweezer technology in accordance with the present embodiments, multiple mitochondria can be trapped simultaneously (see FIGS. 8B, 9A, 9B, 10A, 10B). As the optical tweezer system in accordance with the present embodiments outputs a maximum power of 3 W and each mitochondrion requires about 0.5 W to capture, up to six mitochondria can be transported and captured at the same time. The graph 1530 depicts reconstruction of the cell after transfer of four mitochondria, and the graph 1560 depicts reconstruction of the cell after transfer of six mitochondria.

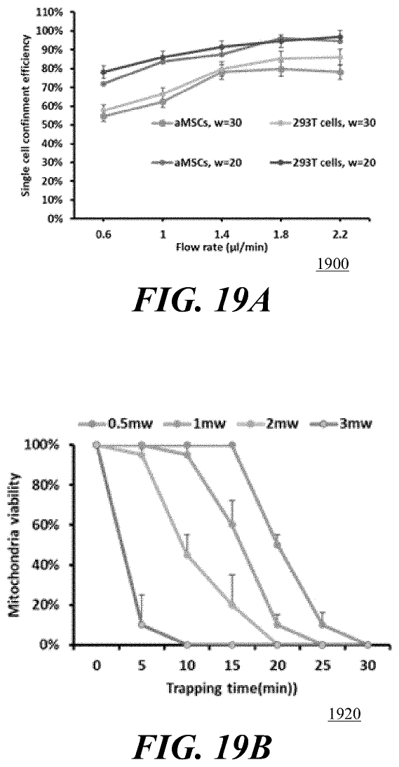

Referring to FIGS. 16A to 16F, images 1600, 1610, 1620, 1630, 1640, 1650 depict the experimental results of transferring three mitochondria isolated from fMSC to an aMSC. The image 1600 depicts the confined aMSC. The procedures included trapping three mitochondria in parallel as shown in the image 1610, bringing these mitochondria close to one another to facilitate transport, and raising them by 20 μm as shown in the image 1620 before transporting the three mitochondria to the middle of the cell-confinement channel above the confined cell as shown in the image 1630. Finally, the three mitochondria are released on the cell surface as shown in the image 1640 to achieve successful transfer of two mitochondria in an aMSC as shown in the image 1650.

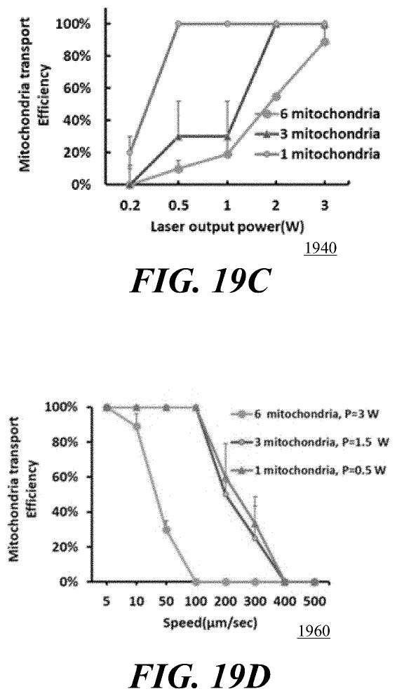

Referring to FIGS. 17A and 17B, six fMSC-isolated mitochondria are transferred to an aMSC as shown in images 1700, 1710, 1750. In the image 1700, the six mitochondria are shown after absorption into the aMSC. Before mitochondrial transfer, the recipient aMSC was stained with MitoTracker green to highlight its endogenous mitochondria as shown in the image 1710. The white arrows in the image 1750 indicate the six transferred mitochondria present in different locations within aMSC. The transferred mitochondria were located together with the endogenous mitochondria of aMSC, indicating the successful endocytosis of the isolated exogenous mitochondria.

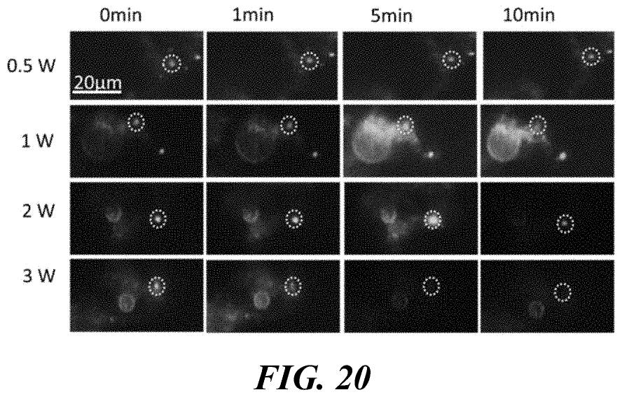

The efficiency of OT-based mitochondrial transfer was evaluated using different cell types and different cell-confinement channel heights in the microfluidic device 102, as shown in the bar graph 1800 of FIG. 18A. Mitochondria-transfer efficiency is defined as the ratio of the number of mitochondria successfully absorbed by the cell to the number of mitochondria transported onto the top of the cell with optical tweezer system in accordance with the present embodiments. As shown in the bar graph 1800, an average mitochondria-transfer efficiency was 42% for aMSC and 26% for 293T cells, averaging the bars for channel height (h) equals 50 μm and 25 μm (n=223 and 280 for aMSC when channel height h=50 μm and h=25 μm, respectively; n=211 and 253 for 293T cells when h=50 μm and h=25 μm, respectively). The efficiency is primarily affected by the transport efficiency of mitochondria via the optical tweezer system 104, the size of the cell-confinement channels 124, and the endocytosis capacity of the recipient cells. Note that the smaller size of the confinement channel ensures that the interaction of the transported mitochondria on the cell surface is improved, thereby increasing the chance of mitochondrial uptake. Conversely, a larger size of the cell-confinement channel may cause the mitochondria to leave the channel quickly. The typical size of 293T cells and aMSCs during suspension ranged within 16-19 μm (horizontal and vertical). When the size of the channel is 25 μm (height)×20 μm (width), mitochondria can be successfully transported onto the top of the cell because the diameter of the cell is typically less than 20 μm.

Referring to FIG. 18B, a bar graph 1850 indicates that a low flow rate (e.g., 1 μL/min) during the cell hydrodynamic confinement process can ensure high cell viability (>85%). At low flow rates, single-cell confinement and the mitochondrial transfer process did not greatly affect cell viability. The efficiency of single-cell confinement was defined as the ratio of the number of single cells confined in the microfluidic device to the total number of single-cell-confinement channels. Cell-confinement efficiency depended on the fluid flow rate in the Inlet 1 106 during cell confinement and the width of the cell confinement U-shaped channel 124 as compared with the cell diameter (n=384±10 cells for all groups, fluid velocity=1 μL/min for all groups, six mitochondria transported in the transferred group, and the size of confinement channel was 20 (w)×25 (h)). The terms “Pass,” “Confined,” and “Transferred” in the bar graph 1850 represent the group of cells that passed through the middle channel (“Pass”), confined in the U-shaped channels 124 (“Confined”), and transferred with mitochondria in the microfluidic device (“Transferred”), respectively.

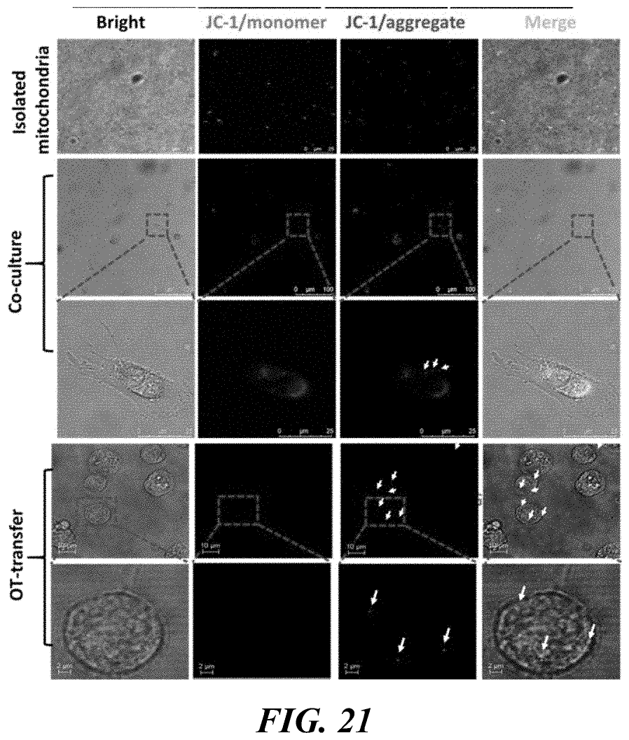

Graph 1900 of FIG. 19A shows the results of single-cell-confinement experiments of two cell types (aMSC and 293T cell) in the U-shaped cell-confinement channels 124 with two different widths (i.e., 30 μm (w) and 20 μm (w)) at different flow rates (n=256 cells for all tests). Higher flow rates tended to push the cells quickly into the empty cell-confinement channels 124, which may damage the cells. A lower flow rate may cause cells to stick near the boundary of the initially confined channel 124 and disrupt the flow of cells behind. When the size of the confinement channel 124 is too large, more than one cell may be confined to a single channel. Therefore, to achieve efficient single-cell hydrodynamic confinement, the width of the U-shaped cell-confinement channel 124 should be equal to or smaller than the cell diameter.

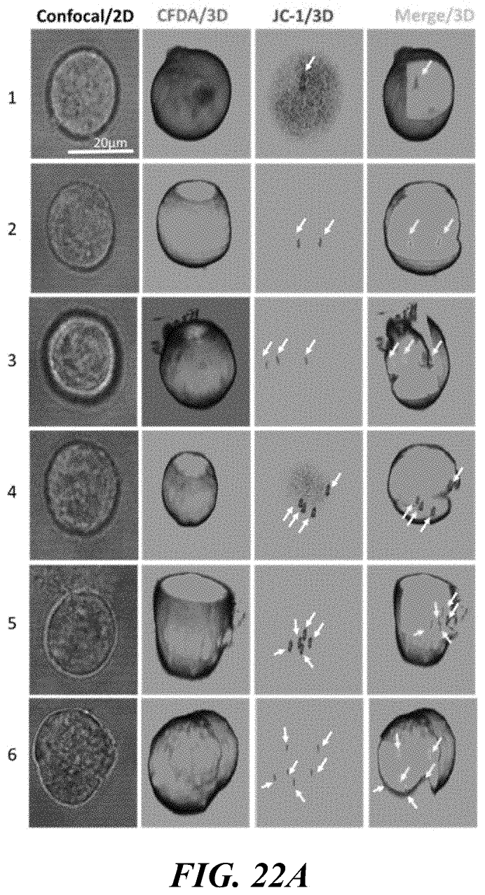

Graph 1920 of FIG. 19B shows the results of mitochondrion-viability tests under different optical tweezer system laser powers and different trapping-time intervals (n=10 mitochondria under each trapping power) and details of the mitochondrion-viability tests are examined hereinbelow in the discussion of FIG. 20. Mitochondria-transport efficiency was defined as the ratio of the number of mitochondria successfully transported onto the cell to the total number of mitochondria selected for transport, and largely depended on the trapping power of optical tweezer system 104 and the fluidic friction of the media during mitochondrial transport. When multiple traps were simultaneously generated with the optical tweezer holographic technology, the power of each optical tweezer trap was determined as the total output power divided by the number of optical traps generated. For example, when the total output power was 3 W and the number of optical tweezer traps was six, the output power of each optical tweezer trap was about 0.5 W. Note that a small amount of laser power may be lost depending on the efficiency of the spatial light modulator inside the holographic optical tweezer system. The estimated optical tweezer system 104 power at the sample plane was ˜450 mW, corresponding to the 3W laser output power. The experimental results show that similar mitochondria-transport efficiency can be achieved when a single optical tweezer with 0.5 W output power was used to transport a single mitochondrion, or when 3 W output power was used to transport six mitochondria in parallel.

Referring to FIGS. 19C and 19D, when using 3 W power to transport six mitochondria in parallel, the mitochondria-transport efficiency was slightly reduced as shown in graphs 1940, 1960, indicating that a small amount of power of the optical tweezer system 104 may be lost. The graph 1940 shows the relationship between laser power and mitochondria-transport efficiency (n=5 tests for each mitochondria group, stage speed=10 μm/s), where laser power means the total optical tweezer system 104 output power applied on one, three, or six mitochondria. The graph 1960 shows that mitochondria-transport efficiency also decreased with increased transport speed (n=5 tests for each testing group, the average laser power of each mitochondrion was ˜0.5 W). A lower transport speed, however, requires a longer processing time to complete the mitochondrial transport of all cells. The speed of the motorized stage for the experiments discussed hereinabove was set to 10 μm/s; at this speed, the system took about ten seconds to transport one mitochondria and fifty seconds to transport six mitochondria in parallel. The entire transport process includes mitochondria detection, cell detection, mitochondrial trapping and transport, and mitochondrial release. Considering 128 cell-confinement channels 124 in the microfluidic device 102, the system takes around 0.35-1.7 hours (depending on the number of mitochondria) to transport all mitochondria. Afterwards, the cells are given another one hour to absorb the transported mitochondria.

Based on the above experimental analysis, TABLE 1 lists the operational parameters of the microfluidic device 102 and the mitochondria-transport process in accordance with the present embodiments.

TABLE 1

| | | | Cell- | | |
|---|---|---|---|---|---|
| | Inlet 1 | | confinement | | |
| | (flow | Inlet 2 | channel size | OT power | Stage |
| | rate) | (flow rate) | (height × | (W)/ | speed |
| Action | μl/min | μl/min | width; μm2) | mitochondria | (μm/s) |
| Single cell | 1 | 0 | 25 × 20 | 0 | 0 |
| confinement | | | | | |
| Mitochondria | 0.2 | 0.5 | 25 × 20 | 0 | 0 |
| placement in | (buffer) | (mito- | | | |
| middle | | chondria) | | | |
| channel | | | | | |
| Mitochondrial | 0 | 0 | 25 × 20 | 0.5/mito- | 10 |
| transport | | | | chondria | |

Based on the above experimental results, the optimal flow rate for single cell confinement was selected as 1 μl/min for the Inlet 1 106 and 0 μl/min for the Inlet 2 108 (i.e., the Inlet 2 108 was blocked). Similarly, the flow rates of the Inlets 1 and 2 for introducing mitochondria into the microfluidic device 102 were selected as 0.2 μl/min for buffer flow into the Inlet 1 106 and 0.5 μl/min for mitochondria flow into the Inlet 2 108, respectively. The size of the single cell confinement channel, the power of the optical tweezers and the speed of the stage were selected to be 25 μm (height)×20 μm (width), 0.5 W/mitochondrion and 10 μm/sec, respectively.

Unlike previous mitochondrial transfer methods that ignore the functional status of isolated mitochondria during transfer, the methods and systems in accordance with the present embodiments can advantageously perform quality and quantity control of mitochondria before transfer. JC-1 dye (obtainable from Thermo Fisher) is a membrane-permeable fluorescent dye usually used to monitor the health of mitochondria. Under a microscope, JC-1 stained mitochondria with high membrane potential (functional mitochondria) typically show red or orange fluorescence, while those with low membrane potential (non-functional mitochondria) show green fluorescence. The fluorescence emission of JC-1 dye occurs at two wavelengths (both excited at 488 nm). Therefore, to check the function of the isolated mitochondria before transfer, the mitochondria of the donor cells were stained with JC-1 before isolation.

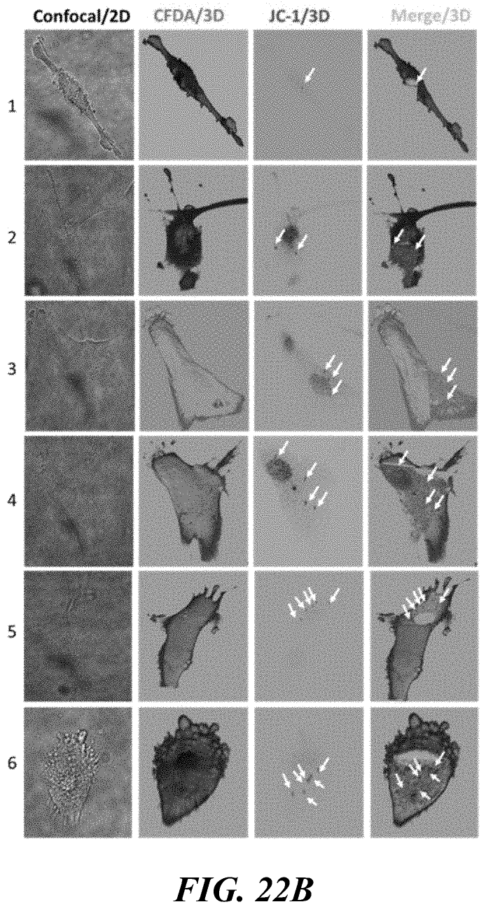

Referring to FIGS. 20 and 21, experimental results show quality control of mitochondrial transfer achieved using the automated optical tweezer-based mitochondrial-transfer system and methods in accordance with the present embodiments. FIG. 20 shows the functional status of mitochondria trapped by the optical tweezer system 104 under different laser power and exposure time (marked with white dotted circles). Under a trapping power of 0.5 W, the mitochondrial membrane potential remained high (orange) for up to ten minutes under continuous exposure. However, when a single mitochondrion was trapped with 2 W power for five minutes, a very obvious change in the mitochondria membrane potential from orange to green was observed. With a trapping power of 3 W, mitochondria lose function after only one minute of exposure. It has been reported that mitochondrial function may be impaired due to the heat of optical tweezer lasers. This effect can be minimized by appropriate control of the laser power and the exposure time. The results shown in FIG. 20 advantageously indicate that 0.5 W could be used to transport individual mitochondria in accordance with the present embodiments without compromising mitochondrial function.

FIG. 21 shows a comparison of the quality control of mitochondrial transfer in 293T cells between a traditional co-cultured method and the optical tweezer-based methods in accordance with the present embodiments. The images of FIG. 21 are confocal and fluorescence microscopy images using a Green Fluorescent Protein (GFP) filter and showing the presence of functional (orange or red) and non-functional (green) 293T-isolated mitochondria stained with mitochondrial membrane potential dye JC-1 and transferred in 293T cells. The cells in the “Co-culture” group were cultured in a petri dish and directly used for imaging after mitochondrial transfer. For the cells in the “OT-transfer” group, imaging was performed shortly after the cells were collected from the microfluidic device 102 and placed in a petri dish. The first row of images of FIG. 21 shows confocal images of the isolated mitochondria, indicating the presence of functional (orange or red (aggregate)) and non-functional (green (monomer)) mitochondria. The second and third rows (represented by “Co-culture”) show mitochondria isolated from 293T cells after they have been transferred in the 293T cells by using the co-culture method. The column labeled “Merge” shows the combined imaging of all the images on the left side of the same row. There are a large number of non-functional green (monomeric) mitochondria in the transferred cells.

The last two rows of images in FIG. 21 denoted by “OT-transfer” show mitochondria isolated from 293T cells after having been transferred in the 293T cells using the optical tweezer-based methods in accordance with the present embodiments. It can be seen that only orange-red mitochondria were observed in the transferred cells, and the number of non-functional (green) mitochondria in the cells was significantly reduced. Thus, the optical tweezer-based mitochondrial-transfer system and methods in accordance with the present embodiments advantageously provide improved quality control over conventional mitochondrial-transfer system and methods, such as the conventional co-cultured method.

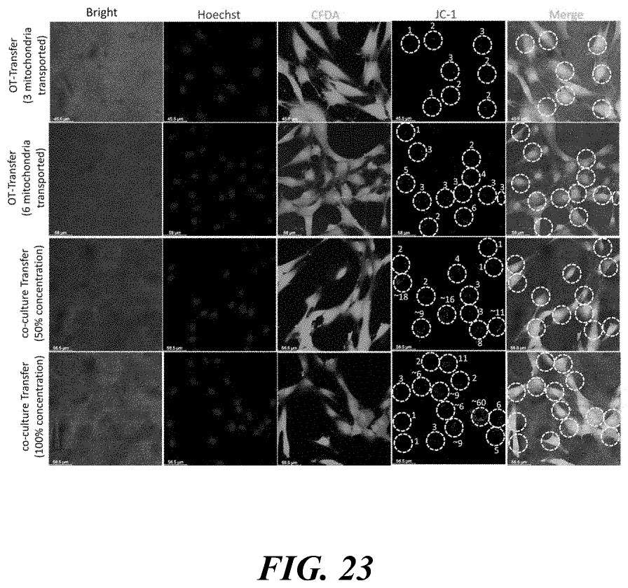

FIGS. 22A and 22B show the advantageous quantity control of mitochondrial transfer achieved by the optical tweezer-based mitochondrial-transfer system and methods in accordance with the present embodiments. FIGS. 22A and 22B show confocal three-dimensional images of different numbers (one to six shown on the left side of each row) of mitochondria transferred in 293T cells and aMSCs, respectively. The cross-transfer of mitochondria was also investigated and advantageously demonstrated that isolated mitochondria obtained from different sources can be successfully transferred in different cell types. The column represented by “Confocal/2D” shows bright-field images of mitochondrial transferred cells. The column represented by “CFDA/3D” and “JC-1/3D” respectively show three-dimensional confocal images of cells stained with CFDA (for 3D cell visualization) and JC-1 dye staining (for transfer visualization of mitochondria). The column represented by “Merged/3D” shows images of different numbers (1-6) of mitochondria successfully internalized.

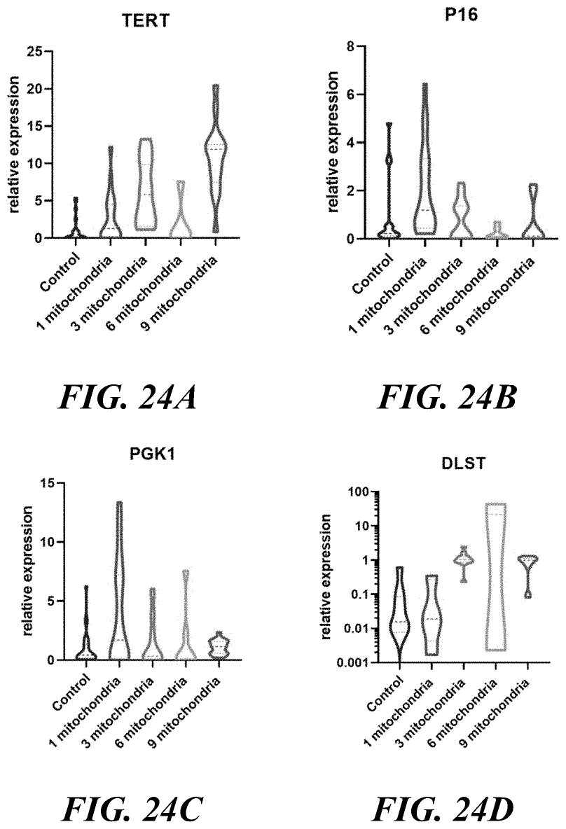

Referring to FIG. 23, shows a comparison of quantity control between the optical tweezer-based methods in accordance with the present embodiments and the conventional co-culture method. The numbers of mitochondria transported by the optical tweezer-based methods in accordance with the present embodiments are shown on the left side of the first and second rows. Each dotted circle in the last two columns indicates an individual cell with transferred mitochondria and the number on top of each circle indicates the number of successful mitochondrial transfer in each cell. The numbers on top of dotted circles show the number of mitochondria transferred in each cell. In the co-culture mitochondrial transfer method in the last two rows, mitochondria is isolated from 10⁷ fMSCs and divided into two concentrations (1:2). Each isolated mitochondrial mixture was co-cultured with aMSCs cultured in a petri dish and placed in an incubator for 1.5 hours.

The first and the second rows of FIG. 23 show the results of mitochondrial transfer in aMSCs by the optical tweezer-based methods in accordance with the present embodiments. When three and six mitochondria were transported onto the top of the cell, the number of mitochondria absorbed by the cell varied between zero to three and zero to six, respectively, indicating that the upper limit of mitochondria transferred in cells can be controlled. The third and fourth rows in FIG. 23 show the results of mitochondrial transfer in aMSCs through co-culture methods of different concentrations, where the upper limit of mitochondrial transfer cannot be controlled using co-culture methods.

All the experimental results discussed hereinabove indicate that the optical tweezer-based methods in accordance with the present embodiments advantageously has the ability to control the quality and quantity of the transfer of mitochondria to cells. This can be achieved by successfully patterning cells and isolated mitochondria in the microfluidic device 102, and then automatically detecting and transporting mitochondria.

Mitochondria primarily control energy metabolism in cells. The secretome of fMSCs can improve the aging phenotype of aMSC in in-vitro and in-vivo models, thus indicating that fMSCs may have a unique metabolic profile in the secretome and have anti-aging ability.

A senescent cell usually shows increased glycolysis activity owing to mitochondria deficiency. Thus, the high activity of gluconeogenesis suggests that mitochondria of fMSCs may exert a unique metabolic regulation effect, thereby reducing aging phenotype.

Through mitochondrial transfer from fMSCs to aMSCs, the metabolic genes in aMSCs can also become highly expressed, indicating that the phenotype of aMSC can become fMSC-like.

Telomerase (TERT) is a recognized anti-aging gene. The increase in TERT activity indicates that cell status improved in terms of cell proliferation, differentiation, lifespan, and other basic cell physiology.

In cellular senescence, P16 is a key indicator of cell-cycle inhibition during aging. After mitochondrial transfer, the decrease in P16 expression indicates that the transferred cells had increased proliferation ability, which is also supported by high KI67 expression, a proliferating cell-specific membrane antigen.

FIGS. 24A to 24D shows the single-cell qPCR results of aMSCs transferred with different numbers of fMSC-isolated mitochondria. FIG. 24A depicts expression levels of TERT, FIG. 24B depicts expression levels of P16, FIG. 24C depicts expression levels of PGK1, and FIG. 24D depicts expression level of DLST. The gene expression of key genes such as TERT, PGK1, and DLST increased after transfer, as shown in FIGS. 24A, 24C and 24D. The number of transferred mitochondria affected the expression levels of genes. Compared with the control group, after six mitochondrial transfers the expression of TERT, PGK1, and DLST1 increased, whereas the expression of P16 decreased (indicating that cell proliferation was activated). Interestingly, the transfer of one or three mitochondria have also induced significant changes in gene-expression levels. For example, TERT and PGK1 were activated two hours after the transfer of only one mitochondria, indicating that the transfer of a small number of mitochondria from fMSC may be sufficient to activate the metabolic activity of aMSC. It is also noted that not all expression levels of genes, such as that of PGK1, increased with the increased number of transferred mitochondria.

Thus, it can be seen that the methods and systems in accordance with the present embodiments, an automated optical tweezer-based manipulation approach was developed for mitochondrial transfer in single cells. A microfluidic cell-positioning device was used to pattern cells and mitochondria, and an optical tweezer manipulation system was used to collect a predefined number of healthy isolated mitochondria and transport them onto the top of the cells automatically. Then, the cells absorbed these mitochondria through endocytosis.

In contrast to the passive transfer method by co-culture, the methods and systems in accordance with the present embodiments efficiently controls the quantity and quality of the mitochondria before transfer. Results of cell anti-aging and metabolic gene expression, examined using qPCR analysis, indicate that the mitochondria of fMSCs have the potential to reverse the aging phenomenon in aMSCs. The methods and systems in accordance with the present embodiments can considerably contribute to precision medicine and cell therapy of mtDNA-related diseases.

Table 2 compares the performances of the method and system in accordance with the present embodiments with other existing methods. Microneedle-based methods (such as microinjection and thermal nanoblades) are fast but are limited by efficiency, the number of mitochondrion injections, and the inability to control the quality and quantity of mitochondrial transfer. The co-culture-based mitochondrial transfer method is efficient, moderately fast (depending on the recipient cell), and can obtain therapeutic amounts of mitochondrial transfer cells for clinical use. However, this method cannot control the quality and quantity of mitochondrial transfer in recipient cells. The optical tweezer-based mitochondrial transfer methods and systems in accordance with the present embodiments is non-invasive, efficient, moderate speed, and can accurately select a specific number of healthy mitochondria and then transport them to specific single cells. Further transfer of these mitochondria within the cell depends on the endocytosis of the cell. Some studies have reported the use of external forces (such as magnetic fields or fluid pressure) to push mitochondria into cells.

It should also be noted that the optical tweezer-based mitochondrial transfer methods and systems in accordance with the present embodiments can achieve quantity control of mitochondria transfer to a certain extent. That is, the number of mitochondria can be controlled within a range having a clear upper limit of mitochondria (equal to the number of mitochondria transported on each cell via the optical tweezer system 104), but it is difficult to determine the lower limit of mitochondria because this depends on the endocytosis of the cell.

TABLE 2

| | | Quality | | |
|---|---|---|---|---|
| | | and | | |
| | | quantity | | Number of |
| | | control | Toxicity or | mitochondria |
| Technique | Efficiency | ability | damage | transferred |
| Co-cultured | 0.2%-28% | No | Internalization | No specific |
| isolated | | | of cell debris or | number, |
| mitochondria | | | dead | depends on |
| | | | mitochondria | endocytosis |
| | | | | ability of the |
| | | | | recipient cells |
| Tunneling | 2%-15% | No | — | — |
| nanotube | | | | |
| Microinjection | 0.2%-0.3% | No | Physical damage | One to three |
| | | | to the cell and | |
| | | | injection of | |
| | | | buffer media | |
| Photothermal | 2% | No | Physical damage | One to three |
| nanoblade | | | to the cell by | |
| | | | laser and | |
| | | | injection of | |
| | | | buffer media | |
| Magneto- | | No | Toxicity owing | No specific |
| mitotransfer | | | to | number, |
| | | | internalization | depends on the |
| | | | of metallic | concentration of |
| | | | beads | mitochondria |
| Pressure-driven | | No | Disruption of | No specific |
| | | | plasma | number, |
| | | | membrane and | depends on the |
| | | | injection of | concentration of |
| | | | buffer media in | mitochondria |
| | | | cells | and applied |
| | | | | pressure |
| Optical | 26%-42% | Yes | Damage to | One to nine |
| tweezer-based | | | mitochondria | |
| method in | | | when OT | |
| accordance | | | trapping time is | |
| with the | | | >10 min or OT | |
| present | | | output power is | |
| embodiments | | | >0.5W/ | |
| | | | mitochondria | |

All of the methods shown in TABLE 2 transferred isolated mitochondria into cells. Obtaining a high yield of 100% pure functional isolated mitochondria is difficult due to many reasons, such as the existence of other organelles with similar size as the mitochondria in cells, the long-time isolation process and the external environmental effects on isolated mitochondria. Consequently, isolated mitochondria may contain cell debris and dead mitochondria, which are potentially harmful to host cells. Moreover, the impurities of isolated mitochondria can cause the transfer of debris and dead mitochondria (due to the long duration of the isolation process) into the cells, which may harm the host cells. Yet the optical tweezer-based mitochondrial transfer methods and systems in accordance with the present embodiments can control not only the quantity of mitochondria transferred, but also the quality of mitochondria transferred.

The present embodiments relate to the development of a unique approach of mitochondria transfer in single cells automatically using a robot-assisted optical tweezer micromanipulation system. As seen in the discussion herein, existing mitochondria transfer methods ignore mitochondrial heterogeneity, including mitochondrial functionality that represents another level of mitochondrial complexity. Compared with the existing mitochondria transfer techniques, the mitochondrial transfer method and system in accordance with the present embodiments detects only healthy isolated mitochondria using image processing and then transfer them to specific single cells.

The number of mitochondria to be transferred is an important aspect of precision medicine. The precise quantity-controlled mitochondria transfer method and system in accordance with the present embodiments can help find answers to some open questions like; how many transferred mitochondria will be enough to make a significant change in a single cell? Compared to existing mitochondria transfer methods, the method and systems in accordance with the present embodiments allow a predefined number of mitochondria to be accurately collected, transported and transferred to each cell.

The experimental results show that, unlike the previous mitochondrial transfer methodologies that ignored the functionality status of isolated mitochondria during their transfer, the optical tweezer-based mitochondrial transfer methods and systems in accordance with the present embodiments can advantageously control the quality and quantity of the mitochondria prior to transfer. Existing mitochondria transfer methods ignore mitochondrial heterogeneity, including mitochondrial functionality. The optical tweezer-based mitochondrial transfer methods and systems in accordance with the present embodiments enables automatic and precise collection and transport of healthy mitochondria onto cells with high efficiency, where the cells uptake these healthy mitochondria through endocytosis. The number of mitochondria to be transferred is an important aspect in precision medicine and the exact number of mitochondria that could cure or cause a significant change in single cells for a particular disease is completely unknown. Thus, the precise quality-controlled optical tweezer-based mitochondrial transfer methods and systems in accordance with the present embodiments which allows predefined number of mitochondria to be accurately collected, transported and transferred to each individual cell is a highly necessary element of precision medicine.

While exemplary embodiments have been presented in the foregoing detailed description of the present embodiments, it should be appreciated that a vast number of variations exist. It should further be appreciated that the exemplary embodiments are only examples, and are not intended to limit the scope, applicability, operation, or configuration of the invention in any way. Rather, the foregoing detailed description will provide those skilled in the art with a convenient road map for implementing exemplary embodiments of the invention, it being understood that various changes may be made in the function and arrangement of steps and method of operation described in the exemplary embodiments without departing from the scope of the invention as set forth in the appended claims.

What is claimed is:

1. A system for mitochondria transfer onto cells, the system comprising:
one or more confinement means for confining cells;
means for locating mitochondria near the one or more confinement means; and
an optical tweezer micromanipulation system configured to trap at least one of the mitochondria for transport of the at least one of the mitochondria to one of the confined cells.

2. The system in accordance with claim 1 further comprising:
a microfluidic device comprising the one or more confinement means and a channel, the channel comprising the means for locating the mitochondria near the one or more confinement means; and
at least one positioning stage, wherein the microfluidic device is mounted on the at least one positioning stage for movement of the microfluidic device in relation to the optical tweezer system, and wherein the optical tweezer micromanipulation system is configured to trap at least one of the mitochondria within the channel for transport of the at least one of the mitochondria to one of the confined cells.

3. The system in accordance with claim 1 wherein the microfluidic device further comprises at least one fluid inlet fluidically coupled to the channel and configured to flow the mitochondria into the channel.

4. The system in accordance with claim 1 wherein the confinement means comprises one or more single-cell confinement channels, the shape and dimensions of the one or more single-cell confinement channels configured to correspond to dimensions of the cells to be confined.

5. The system in accordance with claim 4 wherein the microfluidic device further comprises at least one fluid outlet, the at least one outlet fluidically coupled to the one or more single-cell confinement channels for confining the cells within the one or more single-cell confinement channels.

6. The system in accordance with claim 5 wherein a plurality of the one or more single-cell confinement channels are formed into a one-dimensional array of single-cell confinement channels, and wherein the one-dimensional array of single-cell confinement channels is fluidically coupled to the at least one outlet for confining the cells within the one-dimensional array of single-cell confinement channels.

7. The system in accordance with claim 2 further comprising automation control means coupled to the optical tweezer micromanipulation system and the at least one positioning stage and configured to activate the optical tweezer micromanipulation system to trap the at least one of the mitochondria, the automation control means further configured to move the microfluidic device in relation to the optical tweezer micromanipulation system to transport the at least one of the mitochondria to the one of the confined cells and place the at least one of the mitochondria on a surface of the one of the confined cells.

8. The system in accordance with claim 7 further comprising imaging means optically coupled to the microfluidic device, wherein the imaging means is coupled to the automation control means and configured to generate an image of the at least one of the mitochondria, and wherein the automation means is configured to determine from the image of the at least one of the mitochondria whether the at least one of the mitochondria is a healthy isolated mitochondria and activate the optical tweezer micromanipulation system to trap the at least one of the mitochondria when the at least one of the mitochondria is determined to be a healthy isolated mitochondria.

9. The system in accordance with claim 7 wherein the at least one positioning stage is configured to move the microfluidic device in three dimensions, and wherein the automation control means is configured to move the microfluidic device in relation to the optical tweezer micromanipulation system to raise the at least one of the mitochondria in the channel of the microfluidic device, to transport the at least one of the mitochondria within the channel to the one of the confined cells, and to lower the at least one of the mitochondria to place the at least one of the mitochondria on the surface of the one of the confined cells.

10. A method for transporting mitochondria to cells for absorption by the cells, the method comprising:
trapping the mitochondria by an optical tweezer micromanipulation system;
transporting the mitochondria over a confined cell; and
placing the mitochondria on a surface of the confined cell so that the confined cell can absorb the mitochondria by endocytosis.

11. The method in accordance with claim 10 further comprising confining the cell in a single-cell confinement channel before trapping the mitochondria.

12. The method in accordance with claim 10 further comprising flowing the mitochondria nearby the confined cell before trapping the mitochondria.

13. The method in accordance with claim 10 further comprising determining whether the mitochondria is a healthy isolated mitochondria before trapping the mitochondria.

14. The method in accordance with claim 10 further comprising determining a center of the confined cell before transporting the mitochondria over the confined cell.

15. The method in accordance with claim 14 wherein determining the center of the confined cell comprises determining a center of a single-cell confinement channel confining the confined cell.

16. The method in accordance with claim 12 wherein flowing the mitochondria comprises flowing the mitochondria nearby the confined cell within a channel of a microfluidic device, and wherein transporting the mitochondria over the confined cell comprises:
raising the optical tweezer micromanipulation system to raise the mitochondria within the channel to above a level of the confined cell;
moving the microfluidic device to align the mitochondria with a center of the confined cell; and
lowering the optical tweezer micromanipulation system to lower the mitochondria onto the surface of the cell.

17. A computer readable medium containing program instructions for enabling transportation of mitochondria to cells by an optical tweezer micromanipulation system for absorption by the cells, the program instructions when compiled into a processor are configured to cause the processor to:
trap a mitochondria by the optical tweezer micromanipulation system;
transport the mitochondria over a confined cell; and
place the mitochondria on a surface of the confined cell so that the confined cell can absorb the mitochondria by endocytosis.

18. The computer readable medium in accordance with claim 17 wherein the program instructions further cause the computer to:
confine the cell in a single-cell confinement channel and flow the mitochondria nearby the confined cell before trapping the mitochondria.

19. The computer readable medium in accordance with claim 17 wherein the program instructions further cause the computer to:
obtain an image of the mitochondria and determine whether the mitochondria is a healthy isolated mitochondria before trapping the mitochondria.

20. The computer readable medium in accordance with claim 17 wherein the program instructions further cause the computer to:
determining a center of the confined cell by determining a center of a single-cell confinement channel confining the confined cell before transporting the mitochondria over the confined cell.
